(19) United States
(12) Patent Application Publication
(10) Pub. No.: US 2014/0066828 A1
(43) Pub. Date: Mar. 6, 2014

(54) BACK SUPPORT VEST

(71) Applicant: Naipaul Sookdeo, Orange, NJ (US)

(72) Inventor: Naipaul Sookdeo, Orange, NJ (US)

(21) Appl. No.: 14/018,380

(22) Filed: Sep. 4, 2013

Related U.S. Application Data
(60) Provisional application No. 61/743,430, filed on Sep. 4, 2012.

(51) Int. Cl. A61F 5/02

(52) U.S. Cl. CPC A61F 5/02
USPC 602/19

(57) ABSTRACT

A back support vest may include a vest body having a bottom portion, a front portion, a rear inner portion, a rear outer portion, a first side and a second side. The first side may have an arm hole and the second side may have an arm hole. A plurality of padding may be attached to the rear inner portion of the vest body. The vest body may be tightened by a tightening component attached to the rear outer portion of the vest body. A closing component may be attached to the front portion of the vest body. The back support vest may comprise a fastener attached below the arm hole on the first side of the vest body down to the bottom portion of the vest body.

### CROSS-REFERENCE TO RELATED APPLICATION

This application claims the benefit of priority of U.S. provisional application No. 61/743,430, filed Sep. 4, 2012, the contents of which are herein incorporated by reference.

### BACKGROUND OF THE INVENTION

The present invention relates to back support and, more particularly, to a back support vest.

Millions of people suffer with constant back pain, soreness and aching backs. The pain may be while they are walking, sitting at their desk, working or the like. Currently, people will place pillows behind their backs, or have to stop work and lay down on the floor to stop the pain. A brace may be something that a person would wear, but they would have to stay home, losing time from work or school. Black belts are mostly used by men, focus on a specific area, and are not fashionable. White body bandages have to be worn under a person's clothes, so taking them off at work may be awkward or impossible in a public place. Other devices are made of metal or plastic that can hold the neck up, but can only be worn during a home stay with time away from work. Additionally, people may use patches or heated creams at home. These devices may provide relief for specific areas of the body.

As can be seen, there is a need for a back support vest that may be loosened or tightened, worn indoors or out, over clothing or under, that supports various body parts and which a person may take it off and back on easily whenever they like.

### SUMMARY OF THE INVENTION

In one aspect of the present invention, a back support vest comprises: a vest body having a bottom portion, a front outer portion, an inner surface, a rear outer portion, a first side and a second side, wherein the first side and the second side have an arm hole; a plurality of padding attached to the inner surface of the vest body; a tightening component attached to the rear outer portion of the vest body; and a closing component attached to the front outer portion of the vest body.

In another aspect of the present invention, a method for reducing back pain comprises: placing a back support vest over a user's head, wherein the back support vest comprises a vest body having a bottom portion, a front outer portion, an inner surface, a rear outer portion, a first side and a second side, wherein the first side has an arm hole and the second side has an arm hole; placing the user's arms through the two arm holes; closing the front outer portion of the vest body; and tightening the rear outer portion of the vest body.

In another aspect of the present invention, a method for reducing back pain comprises: placing a back support vest over a user's head, wherein the back support vest comprises a vest body having a front outer portion, an inner surface, a rear outer portion, a first side and a second side, wherein the first side has an arm hole and the second side has an arm hole; placing the user's arms through the two arm holes; and closing a fastener below the arm hole of the first side of the vest body.

These and other features, aspects and advantages of the present invention will become better understood with reference to the following drawings, description and claims.

### BRIEF DESCRIPTION OF THE DRAWINGS

FIG. 1 is a front view of an exemplary embodiment of the present invention;

FIG. 2 is a front view of an exemplary embodiment of the present invention unlaced and folded outwards;

FIG. 3 is a side view of an exemplary embodiment of the present invention;

FIG. 4 is a rear view of an exemplary embodiment of the present invention;

FIG. 5 is a section view of an exemplary embodiment of the present invention along line 5-5 in FIG. 1;

FIG. 6 is a side view of an alternate exemplary embodiment of the present invention;

FIG. 7 is a side view of an alternate exemplary embodiment of the present invention; and FIG. 8 is a side view of an alternate exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a back support vest. The back support vest may include a vest body having a bottom portion, a front outer portion, an inner surface, a rear outer portion, a first side and a second side. The first side may have an arm hole and the second side may have an arm hole. A plurality of padding may be attached to the inner surface of the vest body. The vest body may be tightened by a tightening component attached to the rear outer portion of the vest body. A closing component may be attached to the front outer portion of the vest body.

The present invention may support the whole upper torso, upper back, lower back, shoulders, chest and abdominal muscle areas. The back support vest may be worn for a few hours or for a full day or more.

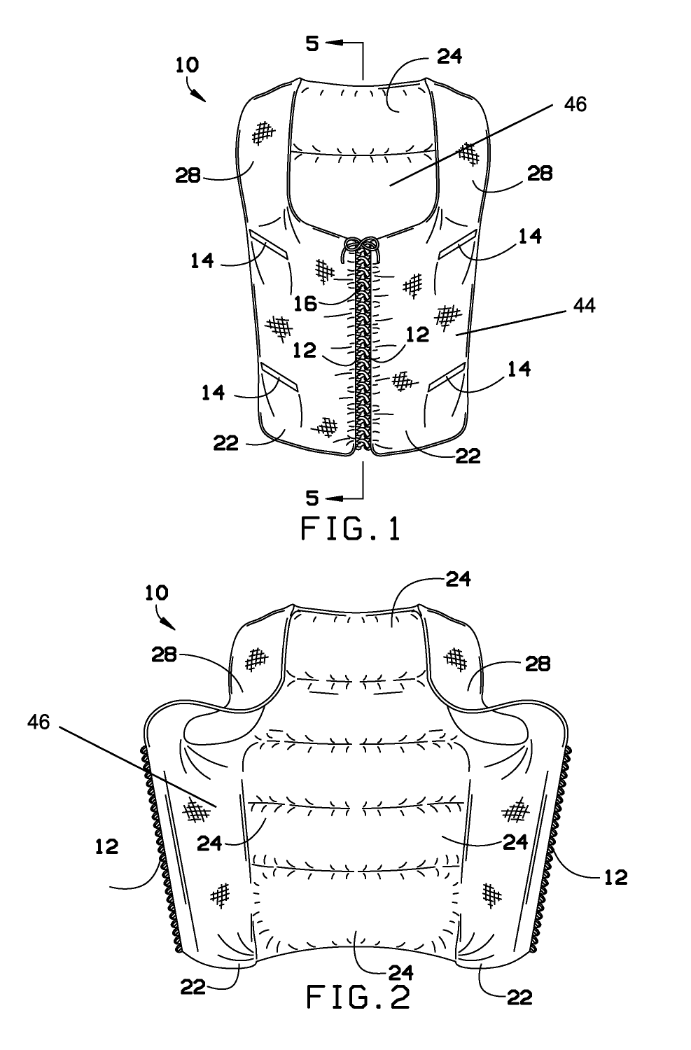

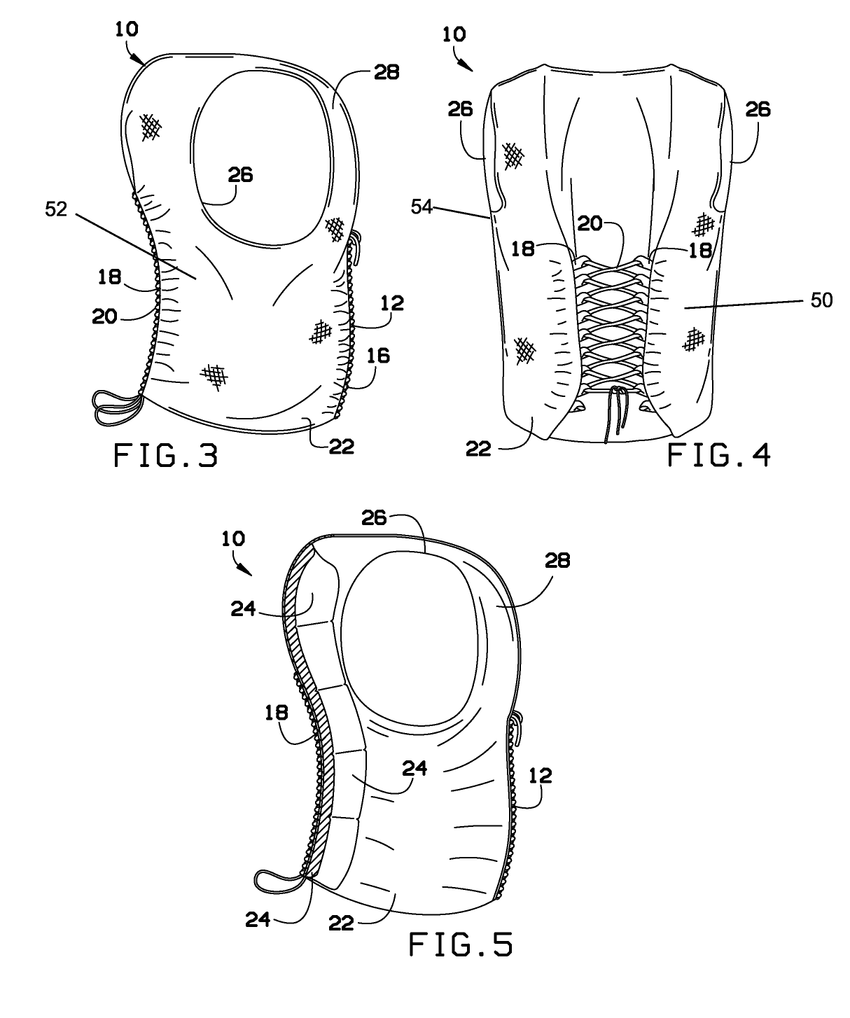

Referring to FIGS. 1 through 8, the back support vest 10 may include a vest body 22. The vest body 22 may have a bottom portion 56, a front outer portion 44, an inner surface 46, a rear outer portion 50, a first side 52 and a second side 54. The vest body may include at least one shoulder strap 28. The shoulder strap 28 may provide an opening for an arm hole 26. A plurality of padding 24 may be attached to the inner surface 46 of the vest body 22.

In certain embodiments, a tightening component may be attached to the rear outer portion 50 of the vest body 22. The tightening component may comprise a lace and loop system where a lace 20, such as a shoelace, may be pulled through a plurality of loops 18 which may be attached to the rear outer portion of the vest body 22 aligned vertically in two columns. The harder the lace 20 is pulled, the tighter the fit of the back support vest. The lace 20 may be tied at the end to present the lace and loop system from opening. The tightening component may be the lace and loop system, a zipper, a hook and fastener system, or the like.

In certain embodiments, a closing component may be attached to the front outer portion 44 of the vest body 22. In certain embodiments the closing component may comprise a lace and loop system. The lace and loop system for the closing component may include a lace 16, such as a shoelace, pulled through loops 12 and then the lace 16 may be tied to prevent the lace and loop system from opening. The closing component may be the lace and loop system, a zipper, a hook and fastener system, or the like. The closing component may connect the first side 52 and the second side 54 which may be separable along the front outer portion 44. In certain embodiments the front outer portion 44 of the vest body 22 may be one piece connecting both the first side 52 and second side 54 of the vest body 22.

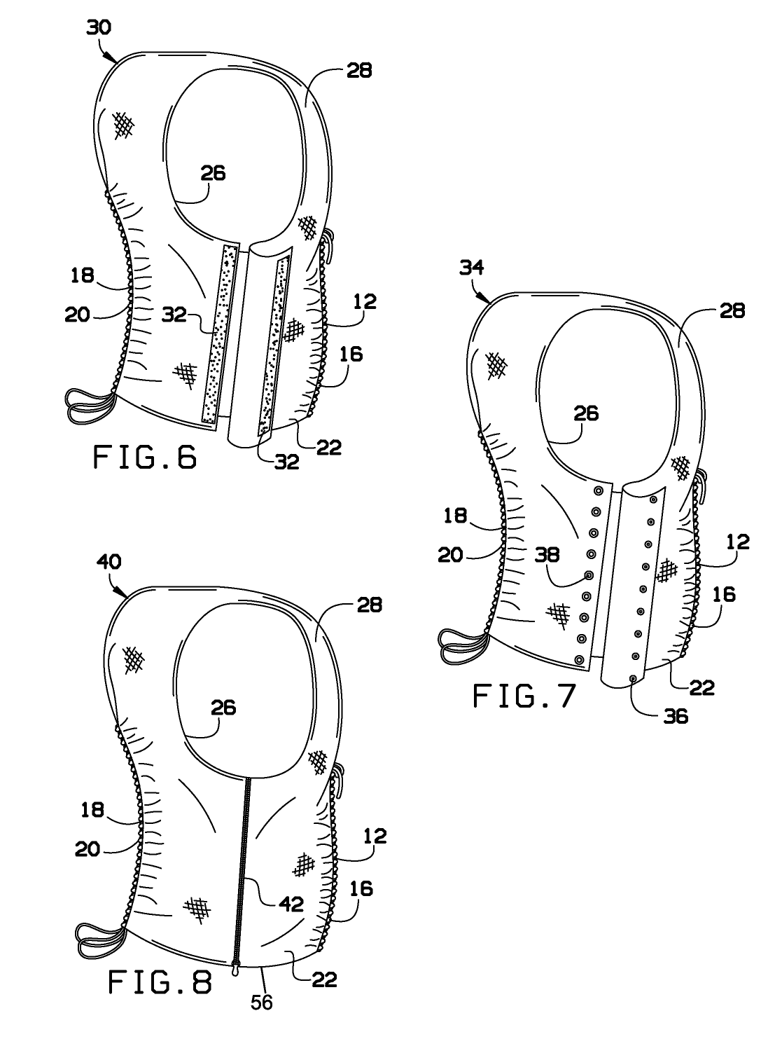

In certain embodiments, a fastener may be attached below the arm hole 26 of the first side 52 of the vest body 22 down to the bottom portion 56 of the vest body. The fastener may be, but not limited to, a hook and loop fastener 32 for a hook and loop fastener embodiment 30. The fastener may be at least one male snap 36 and at least one female snap 38 for a snap fastener embodiment 34. The fastener may be a zipper 42 for a zipper fastener embodiment 40. These particular embodiments may be seen in FIGS. 6 through 8.

In certain embodiments, the back support vest may include at least one pocket 14 attached to the front outer portion 44 of the vest body 22. Additional accessories may be attached to the vest body 22. The back support vest may be made from denim or the like.

A user may place the back support vest over their head and place their arms within the arm holes. The user may close the front outer portion of the vest body. The rear outer portion may be tightened and set. When the user takes the back support vest off, the user may pull the back support vest over their head to remove. In certain embodiments, a user may remove the back support vest by unfastening the fastener, such as the hook and loop fastener, the snap fastener, the zipper fastener or the like, along the first side and pulling the back support vest over the user's head. When removing the back support vest this way, a user may place it back on with the tightness set from the previous wear, and simply refasten the first side once the back support vest is pulled back over the head of the user.

It should be understood, of course, that the foregoing relates to exemplary embodiments of the invention and that modifications may be made without departing from the spirit and scope of the invention as set forth in the following claims.

What is claimed is:

1. A back support vest comprising:
a vest body having a bottom portion, a front outer portion, an inner surface, a rear outer portion, a first side and a second side, wherein the first side and the second side have an arm hole;
a plurality of padding attached to the inner surface of the vest body; and
a tightening component attached to the rear outer portion of the vest body.

2. The back support vest of claim 1, further comprising a closing component attached to the front outer portion of the vest body

3. The back support vest of claim 1, wherein the tightening component comprises a lace and loop system.

4. The back support vest of claim 3, wherein the lace and loop system comprises a lace pulled through a plurality of loops aligned vertically in two columns and attached to the rear outer portion of the vest body.

5. The back support vest of claim 2, wherein the closing component comprises a lace and loop system.

6. The back support vest of claim 1, further comprising at least one pocket attached to the front portion of the vest body.

7. The back support vest of claim 1, further comprising a fastener attached below the arm hole on the first side of the vest body down to the bottom portion of the vest body.

8. The back support vest of claim 7, wherein the fastener comprises a hook and loop fastener.

9. The back support vest of claim 7, wherein the fastener comprises at least one male snap and at least one female snap.

10. The back support vest of claim 7, wherein the fastener comprises a zipper.

11. A method for reducing back pain comprises:
placing a back support vest over a user's head, wherein the back support vest comprises a vest body having a front outer portion, an inner surface, a rear outer portion, a first side and a second side, wherein the first side has an arm hole and the second side has an arm hole;
placing the user's arms through the two arm holes;
closing the front outer portion of the vest body; and
tightening the rear outer portion of the vest body.

12. The method of claim 11, further comprising closing a fastener below the arm hole of the first side of the vest body down to the bottom portion of the vest body.

13. A method for reducing back pain comprises the steps of:
placing a back support vest over a user's head, wherein the back support vest comprises a vest body having a front portion, a rear inner portion, a rear outer portion, a first side and a second side, wherein the first side has an arm hole and the second side has an arm hole;
placing the user's arms through the two arm holes; and
closing a fastener below the arm hole of the first side of the vest body.
